(19) United States
(12) Patent Application Publication
(10) Pub. No.: US 2025/0157663 A1
(43) Pub. Date: May 15, 2025

(54) SYSTEMS AND METHODS FOR AUTOMATED TRIAGING OF ELECTRONIC ORDERED LISTS

(71) Applicant: Optum Services (Ireland) Limited, Dublin (IE)

(72) Inventor: David Alexander DICKIE, Bishopton (GB)

(21) Appl. No.: 18/506,546

(22) Filed: Nov. 10, 2023

(51) Int. Cl. G16H 50/30; G16H 40/67; G16H 50/20; G16H 50/70

(52) U.S. Cl. CPC G16H 50/30; G16H 40/67; G16H 50/20; G16H 50/70

(57) ABSTRACT

A method may include transmitting a request for registration by an individual having a condition to a user device associated with the individual, the request including a subset of data associated with the individual; receiving a confirmation of the subset of medical record data and at least one of a first device identification associated with a first device or a second device identification associated with a second device based on the request; determining a risk probability of an adverse event; assigning a position associated with the individual in an ordered list of individuals having the condition based on the risk probability; monitoring update information from the first device and/or the second device; determining an updated risk probability of an adverse event based on the update information; and assigning an updated position associated with the individual in the ordered list of individuals based on the updated risk probability.

### TECHNICAL FIELD

The present disclosure relates generally to the field of data analytics. Particularly, the present disclosure relates to systems and methods for automatically triaging electronic ordered lists utilizing data analytic and machine learning techniques.

### BACKGROUND

Waiting periods to receive certain types of services or resources, many of which are considered elective, are rising. In some areas of the world, up to 1 in 10 people in a given population are waiting on such types of services or resources. On the other hand, a number of providers or provider systems to manage these inventories of individuals waiting for care is dropping rapidly (precipitously in some areas). Yet, despite this need to maximize time and resources directed toward actually servicing individuals, processes for provisioning service to these individuals, who are kept on waiting lists, is overwhelmingly an entirely manual process.

The techniques of this disclosure relate to problems associated with individuals that have been put in a queue and are waiting, often times indeterminately, for a service or resource from a provider.

Aspects of the present disclosure, however, are defined by the attached claims, and not by the ability to solve any specific problem. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

Examples described herein relate to devices, systems, and methods for automated triaging of electronic ordered lists.

According to some embodiments, a computer-implemented method includes transmitting, by one or more processors, a request for registration by an individual having a condition to a user device associated with the individual, the request including a subset of data associated with the individual; receiving, by the one or more processors, a confirmation of the subset of data and at least one of a first device identification associated with a first device or a second device identification associated with a second device based on the request; determining, by the one or more processors, a risk probability of an adverse event based on at least the subset of data; assigning, by the one or more processors, a position associated with the individual in an ordered list of individuals having the condition based on the risk probability; monitoring, by the one or more processors, update information from at least one of the first device or the second device; determining, by the one or more processors, an updated risk probability of an adverse event based on the update information; and assigning, by the one or more processors, an updated position associated with the individual in the ordered list of individuals based on the updated risk probability.

According to some embodiments, a system includes one or more processors; and at least one memory storing instructions that, when executed by the one or more processors, cause the one or more processors to perform operations including: transmitting a request for registration by an individual having a condition to a user device associated with the individual, the request including a subset of data associated with the individual; receiving a confirmation of the subset of data and at least one of a first device identification associated with a first device or a second device identification associated with a second device based on the request; determining a risk probability of an adverse event based on at least the subset of the data; assigning a position associated with the first individual in an ordered list of individuals having the condition based on the risk probability; monitoring update information from at least one of the first device or the second device; determining an updated probability of an adverse event based on the update information; and updating the position associated with the individual in the ordered list based on the updated probability of an adverse event.

According to some embodiments, a non-transitory computer readable medium stores instructions which, when executed by one or more processors, cause the one or more processors to perform operations comprising: transmitting a request for registration by an individual having a condition to a user device associated with the individual, the request including a subset of data associated with the individual; receiving a confirmation of the subset of data and at least one of a first device identification associated with a first device or a second device identification associated with a second device based on the request; determining a risk probability of an adverse event based on the data; assigning a position associated with the individual in an ordered list of individuals having the condition based on the risk probability; monitoring update information from at least one of the first device or the second device; determining an updated probability of an adverse event based on the update information; and updating the position associated with the individual in the ordered list based on the updated probability of an adverse event.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 is a diagram showing an example of an environment for automated triaging of electronic ordered lists, according to an aspect of the present disclosure.

FIG. 2 is a flow chart showing an example process for provisioning care utilizing automated triaging of a care provisioning electronic ordered list (“provisioning list”), according to an aspect of the present disclosure.

FIG. 3A is a flow chart showing an example process for monitoring individuals' assigned positions in provisioning lists as part of a process of FIG. 2, according to an aspect of the present disclosure.

FIG. 3B is a flow chart showing an example process for assigning individuals updated positions in provisioning lists as part of a process of FIG. 2, according to an aspect of the present disclosure.

FIGS. 4A through 4C are example user interface diagrams that illustrate an example information flow related to example electronic questionnaires, according to an aspect of the present disclosure.

FIG. 5 is an example user interface that illustrates example provisioning lists, according to aspects of the disclosure.

FIG. 6 shows an example machine learning training flow chart.

FIG. 7 illustrates an implementation of a computer system that executes techniques presented herein.

### DETAILED DESCRIPTION

While principles of the present disclosure are described herein with reference to illustrative examples for particular applications, it should be understood that the disclosure is not limited thereto. Those having ordinary skill in the art and access to the teachings provided herein will recognize additional modifications, applications, examples, and substitution of equivalents all fall within aspects of the examples described herein.

Various non-limiting examples of the present disclosure will now be described to provide an overall understanding of the principles of the structure, function, and use of systems and methods disclosed herein for automatically evaluating, in real-time or near real-time, conditions of individuals having assigned positions on electronic ordered lists (“provisioning lists”). Such provisioning lists establish an order by which a specified service or resource will be provisioned to those individuals included in the provisioning list.

At the end of one year in Scotland, a country of approximately 5 million people, there were 474,600 individuals waiting for treatment and approximately 8000 vacancies for various types of healthcare professionals. As mentioned above, despite a significant need to maximize time and resources directed toward actually treating individuals, processes for provisioning care to individuals, who are kept on waiting lists like the above mentioned individuals in Scotland, are overwhelmingly an entirely manual process.

More specifically, management of these waiting lists is often the charge of clinicians who perform the procedures that individuals are on these lists waiting to have. Thus, these clinicians, who define an already limited resource, spend much of their time actively prioritizing patients via consultations and review of patient medical records. In turn, these types of wait-listing triage activities limit the time available for these clinicians to perform procedures and thus extends waiting times for individuals.

In addition, many healthcare systems employ a type of “first come, first serve” principle that widely results in those individuals having the most serious and urgent care needs not necessarily being seen first. Large administrative teams are required to manually manage multiple staff (e.g., performing physician, anesthetist, and theatre nurse) schedules and bed allocations. Human error during this process can often lead to missed appointments, unused beds, and ultimately even longer waiting times for individuals.

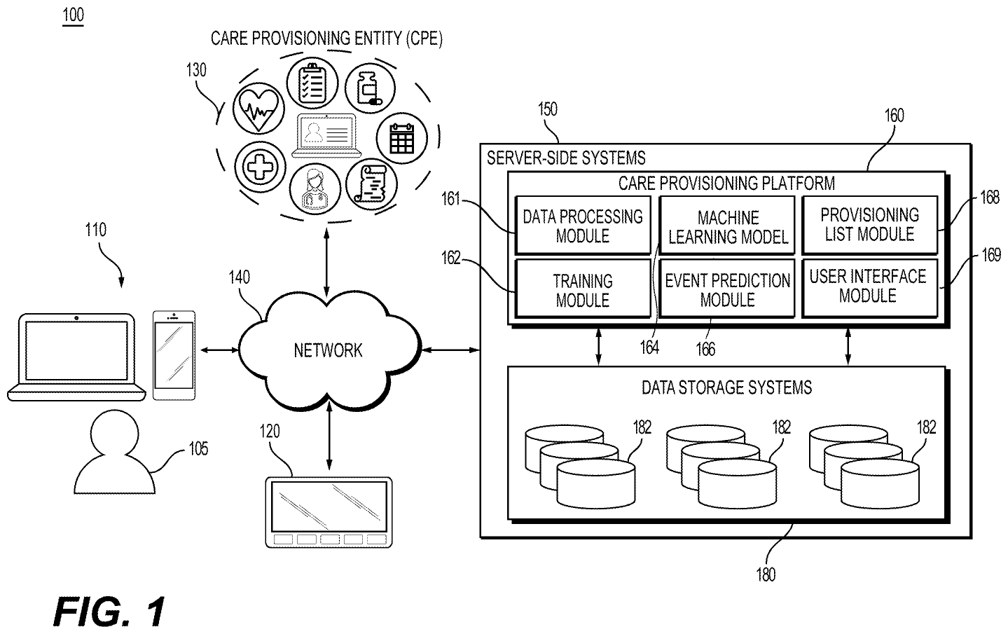

To address these challenges, the techniques of this disclosure enable automatically evaluating, in real-time or near real-time, conditions of individuals and updating individuals' positions in respective queues for care based on updated risk probabilities that the individuals will experience adverse events. Components of an environment 100 of FIG. 1 introduce a capability to implement modern communication and data processing capabilities into methods and systems for dynamically provisioning medical, health, and wellness-related care of limited availability to large groups of individuals, such as, for example, citizens of countries that have national healthcare systems. However, systems and methods described herein are configured to be implemented in any type of system and in essence, distribute out or provision services, professionals, treatments, and other limited resources, to those individuals having the highest risk of experiencing or being subject to a future adverse event potentially due, at least in part, to a current injury or condition, and thus, to those individuals having the most serious and time sensitive needs for the limited resources. In some examples, services, professionals, and other limited resources include: medical doctors; nurses; nurse practitioners; physicians assistants; therapists; physical therapist; bed space; operating time; operating technologies; certain procedures and treatments; and the like.

Although certain embodiments of the present disclosure are described in relation to a healthcare setting, it should be noted that the applicability of the present disclosure is not limited to such a setting. For example, the embodiments of the present disclosure is applicable in other types of industries where automated triaging of ordered lists in anticipation of possible adverse or non-adverse events may be valuable.

FIG. 1 is a diagram showing an example of an environment 100 for automated triaging of electronic ordered lists, according to an aspect of the present disclosure. One or more server-side systems 150 communicate with devices that are associated with requesting individuals (e.g., a user device 110)—the server-side systems 150 and the user device 110 communicating with one or more other components of the environment 100 across a network 140. In some examples, the network 140 facilitates communications between either of the user device 110 and the server-side systems 150, and one or more monitoring devices (e.g., a monitoring device 120) and one or more care provisioning entities (“CPE”) (e.g., a CPE 130). The server-side systems 150 include, define, maintain, or otherwise provide a care provisioning platform 160 and one or more data storage systems 180, among other systems.

In some examples, the user device 110 is used by an individual 105 to communicate with, enroll in, and/or register with example care provisioning platforms, such as the care provisioning platform 160. According to aspects of the present disclosure, individual 105 is a person or a group of people interacting with a user interface or a web interface presented with the user device 110 to access a service, such as an example care provisioning service described herein. In one example, individual 105 includes a registered patient, a potential patient, a returning patient, a visiting patient, an authorized user, etc. that provides contextual information (e.g., health-related information, personal information, etc.) to access the care provisioning service.

In another example, individual 105 completes a registration process using the user device 110 to opt-in to share respective health-related information (e.g., blood pressure, body temperature, skin temperature, heart rate variability, heart rate, resting heart rate, breathing rate, blood glucose, oxygen saturation, stress levels, etc.). In another embodiment, individual 105 shares specialized health indicators (e.g., lab data, blood indicators, physiological data, weight data, etc.). Furthermore, example services and platforms described herein are configured to enable individual 105 to share medication charts, schedule information, medical intake information, calendar appointments, location information, preference information (e.g., specific people as caretakers, healthcare insurance benefit details, etc.).

In one example, the user device 110 includes, but is not restricted to, any type of a mobile terminal, wireless terminal, fixed terminal, or portable terminal. Examples of the user device 110 include, but are not restricted to, a mobile phone/handset, a wireless communication device, a station, a unit, a device, a multimedia computer, a multimedia tablet, an Internet node, a communicator, a desktop computer, a laptop computer, a notebook computer, a netbook computer, a tablet computer, a Personal Communication System (PCS) device, a personal navigation device, a Personal Digital Assistant (PDA), a digital camera/camcorder, an infotainment system, a dashboard computer, a television device, or any combination thereof, including the accessories and peripherals of these devices, or any combination thereof. In addition, the user device 110 facilitates various input means for receiving and generating information, including, but not restricted to, a touch screen capability, a keyboard, and keypad data entry, a voice-based input mechanism, and the like. Any known and future implementations of the user device 110 are also applicable.

By way of example, the user device 110 and/or the monitoring device 120 include one or more sensors that provide any type of sensor. In one example, sensors incorporated therein include, for example, sensors capable of capturing an individual's health data (e.g., activity data, vitals data, blood glucose levels, and any other data that is indicative of the user's health condition) (e.g., inertial measurement unit (IMU) sensors, electrocardiogram (ECG) sensors, sensors to detect blood glucose level, sensors to measure respiration rate, heart rate detection sensors, sensor to monitor body temperature, micro-electro-mechanical system (MEMS) based miniature motion sensors, gyroscope, accelerometer, magnetometer, infrared sensor, camera, microphone, gas sensor, photo-detector, etc.). In another example, sensors included in the user device 110 and/or the monitoring device 120 includes, for example, a network detection sensor for detecting wireless signals or receivers for different short-range communications (e.g., Bluetooth, Wi-Fi, Li-Fi, near field communication (NFC), etc.), a global positioning sensor for gathering location data, a camera/imaging sensor for gathering image data, an audio recorder for gathering audio data, and the like.

In one example, the user device 110 and/or the monitoring device 120 provide a wearable device and/or a health monitoring device, configured to capture parameters such as eating/drinking pattern, exercise regime, medication intake, age, weight, gender, etc. of individual 105. In another example, camera/imaging sensors incorporated in the monitoring device 120 and/or the user device 110 are either a monocular or a stereo camera that captures 3D data of an individual's body (e.g., capture a sequence of images or videos of individual 105 taking prescribed medication).

In one example, the CPE 130 is a service providing entity of one or more types, such as: physicians; nurses; nurse practitioners; healthcare professionals; counselors; medical doctors; wellness professionals; therapeutic or rehabilitation specialists; physical therapists; medical staff; and the like. In one example, the CPE 130 provides medical records, medical history, family/hereditary history, appointment information, or a combination thereof associated with one or more individuals. In another example, the CPE 130 opts-in to share: (i) care provisioning list position information; (ii) provisioned care (e.g., future scheduled appointments, procedures, treatments, counseling, etc.) information; (iii) current scheduling program or portal access; and/or (iv) individuals' files, documents, information, medical history, medication, family/hereditary health history, and/or other health parameters.

In another example, the CPE 130 identifies or requests a set of rules or specialized health indicators to be monitored at specified time intervals by monitoring devices such as the monitoring device 120. In still other examples, the CPE 130 also tags other CPEs as trusted providers, in case of a need for critical, urgent, and/or emergency care by an individual such as individual 105. In addition, the CPE 130 shares information, in anonymous formats in some examples, with the care provisioning platform 160 according to some aspects of the present disclosure. More particularly, the CPE 130 receives requests and/or is required to provide the care provisioning platform 160: details from past appointments, procedures (e.g., surgeries), treatments, counseling sessions; health data including health parameters or diagnosis of individual 105; and/or clinical data for individual 105.

As described in more detail below, the care provisioning platform 160 is configured to process information of the types identified above, as, for example, comparative data for an (adverse) event prediction module 166, and/or library data for the training module 162, and/or input (ground truth input) for the training module 162 and the machine learning model 164. In some examples, incorporations of this information serves as input for implementations of these modules to effect enhancements in overall performance of the server-side systems 150 in ensuring care is provisioned to individuals having the highest risk probabilities for adverse events/most serious and urgent needs, particularly with respect to a current condition, injury, and/or medical, health, or wellness issue defining a primary reason for an individual's placement on a care provisioning list according to the present disclosure.

The user device 110, the monitoring device 120, the CPE 130, and one or more of the server-side systems 150 are connected via network 140, using one or more standard communication protocols. The user device 110, the monitoring device 120, the CPE 130, and the one or more of the server-side systems 150 transmit and receive communications from each other across the network 140. The network 140 is configured to support a variety of different communication protocols and communication techniques, as discussed above. In one example, the network 140 allows the data storage systems 180 and/or the care provisioning platform 160 to communicate with the user device 110, the monitoring device 120, and the CPE 130.

The network 140 of the environment 100 includes one or more networks such as a data network, a wired and/or wireless network, a telephony or cellular network, or any combination thereof. It is contemplated that the data network is any local area network (LAN), metropolitan area network (MAN), personal area network (PAN), wide area network (WAN), a public data network (e.g., the Internet), short range wireless network, or any other suitable packet-switched network, such as a commercially owned (but state accessible), proprietary packet-switched network (e.g., a proprietary cable or fiber-optic network, and the like, or any combination thereof). In addition, the wireless network is, for example, a cellular network and employs various technologies including 5G (5th Generation), 4G, 3G, 2G, Long Term Evolution (LTE), wireless fidelity (Wi-Fi), Bluetooth®, Internet Protocol (IP) data casting, satellite, mobile ad-hoc network (MANET), vehicle controller area network (CAN bus), and the like, or any combination thereof.

Components (e.g., the user device 110, the server-side systems 150) in the environment 100 of FIG. 1 are configured to communicate with each other and other components of the network 140 using well known, new, or still developing protocols. In this context, a protocol includes a set of rules defining how network nodes within the network 140 interact with each other based on information sent over communication links. The protocols are effective at different layers of operation within each node, from generating and receiving physical signals of various types, to selecting a link for transferring those signals, to the format of information indicated by those signals, to identifying which software service, agent, or application executing on a computer system sends or receives the information. The conceptually different layers of protocols for exchanging information over a network are described in the Open Systems Interconnection (OSI) Reference Model.

In some examples, the data storage systems 180 and/or the care provisioning platform 160 are associated with a common entity (e.g., a common CPE, such as a public or national health care system that provides healthcare services to groups of individuals at large). In such examples, the data storage systems 180 and/or the care provisioning platform 160 are part of a cloud service computer system (e.g., in a data center). That is, the various systems can be components or subsystems of a larger computer system. In other examples, one or more of the data storage systems 180 and/or the care provisioning platform 160 are separate systems associated with different entities. In such examples, each of the separate systems are communicatively connected to one another over the network 140 (e.g., via an application programming interface (API)). The systems and devices of the environment 100 can communicate in any arrangement. As will be discussed herein, systems and/or devices of the environment 100 communicate in order to provision care to individuals based on risk probabilities for adverse events and therefore based on need.

Data storage systems 180 and the care provisioning platform 160 each include a server system or computer-readable memory such as a hard drive, flash drive, disk, etc. Data stores 182 of the data storage systems 180 include and/or act as a repository or source for various types of healthcare-related data (e.g., electronic medical record (EMR) data) associated with each of a plurality of individuals. In one example, any of the data stores 182 of the data storage systems 180 are configured to provide any type of database, such as relational, hierarchical, object-oriented, and/or the like, wherein data are organized in any suitable manner, including as data tables or lookup tables. In one example, the data storage systems 180 store and manage multiple types of information providing means for aiding content provisioning and sharing processes implemented by the user interface module 169, in some examples. In another example, the data storage systems 180 include a machine-learning based training database with pre-defined mapping defining a relationship between various input parameters and output parameters based on various statistical methods.

In some examples, the training database includes machine-learning algorithms to learn mappings between input parameters related to an individual such as, but not limited to, physiological parameters, an individual's health records, an individual's lifestyle pattern, etc. and inputs provided by the experts such as, but not limited to, probabilities of adverse events the individual may experience. In an example, the training database includes a dataset that includes data collections that are not subject-specific (e.g., data collections based on population-wide observations, local, regional, or super-regional observations, and the like). Example datasets include environmental information, drug interaction information, geographic data, climate data, meteorological data, retail data, pharmacy data, insurance data, market data, encyclopedias, scientific and medical-related periodicals and journals, business information, research studies data, scientifically-curated genetics-related information, nutritional data, exercise data, physician, and hospital/clinic location information, physician billing information, physician reimbursement information, and the like. In an example, the training database is routinely updated and/or supplemented based on machine learning methods.

The care provisioning platform 160 includes one or more components for automatically evaluating, in real-time or near real-time, health conditions of individuals in order to: (1) determine respective risk probabilities of adverse events; (2) dynamically adjust positions on respective provisioning lists based on the risk probabilities; and (3) ensure certain individuals having the most serious and urgent needs are provided with prioritized access to limited health resources. It is contemplated that functions of these components are combined in one or more components or performed by other components of equivalent functionality. In one embodiment, the care provisioning platform 160 comprises a data processing module 161, a training module 162, a machine learning model 164, an event prediction module 166, a provisioning list module 168, and a user interface module 169, or any combination thereof.

In one example, the data processing module 161 collects relevant data (e.g., health data, health appointment data, behavioral data, contextual data, etc.) associated with individual 105 through various data collection techniques. The data processing module 161 is configured to use a web-crawling component to access various databases (e.g., data storage systems 180 or other information sources to collect relevant data associated with individual 105). In one example, the data processing module 161 collects health data associated with individual 105 via a variety of user devices (e.g., user and monitoring devices 110, 120) that measure physiological parameters (e.g., heart rate, blood oxygen saturation levels, respiratory rate, glucose level, blood pressure, weight, etc.) of individual 105.

In another example, the user device 110 or the monitoring device 120 includes a smartwatch, a smart wristband, a smartphone, smart clothing, or other devices including sensors (e.g., a gyroscope, an accelerometer, a magnetometer, an infrared sensor, a camera, a microphone, a gas sensor, a photo-detector, etc.) capable of capturing activity data and vital data of individual 105. In one example, these monitoring devices are equipped with operating systems like Android™, iOS™, Windows®, Linux™ OS, or hybrid frameworks that enable efficient integration. In one example, the collection of relevant data is automated, e.g., an automatic human activity recognition technique that captures data from wearable and/or non-wearable monitoring devices. The human activity recognition technique is used to build Human Activity Recognition (HAR) datasets. In one example, the data processing module 161 includes various software applications, e.g., data mining applications in Extended Meta Language (XML) that automatically search for and return relevant data regarding individual 105. In one example, the data processing module 161 parses and arranges the data into a common format that is processed by other modules and platforms. In another example, the data processing module 161 collects (e.g., in real-time or near real-time) videos or one or more images of individual 105 from sensors (e.g., image sensors, cameras, etc.) to collect user activity information (e.g., medication intake, exercises, etc.) biometric data (e.g., fingerprints, facial images, etc.).

In one example, the data processing module 161 processes activity data of individual 105 to determine their lifestyle patterns (e.g., eating patterns, drinking patterns, sleeping patterns, exercise patterns, and other activities data such as smoking, drinking, etc.). In another example, the data processing module 161 processes health-related data of individual 105 (e.g., blood pressure, body temperature, skin temperature, heart rate variability, heart rate, resting heart rate, breathing rate, blood glucose, oxygen saturation, or stress levels) to determine changing health conditions. In one example, the data processing module 161 processes health records (e.g., electronic medical records (EMRs)) of individuals to determine their respective health conditions at any given time. In a further example, the data processing module 161 processes calendar information of individual 105 (e.g., patients) and the CPE 130 (e.g., physicians), to determine availability information and potential conflicts in appointments. In another example, the data processing module 161 processes one or more images/videos to determine medication adherence by individual 105.

In one example, the training module 162 provides learning, or training, to the machine learning model 164 by providing training data (e.g., data from other modules such as, e.g., data collected and processed by the data processing module 161, data stored in the data storage systems 180, etc.), that contains input and correct output, to allow the machine learning model 164 to learn over time. The training is performed based on the deviation of a processed result from a documented result when the inputs are fed into the machine learning model 164 (e.g., an algorithm measures its accuracy through the loss function, adjusting until the error has been sufficiently minimized). Training module 162 conducts the training in any suitable manner (e.g., in batches), and includes any suitable training methodology. Training is performed periodically, and/or continuously in real-time or near real-time. Further details of training a machine learning module are provided below.

In one example, the machine learning model 164 receives the training data from the training module 162 to determine a probability of an adverse event for the individual 105. In one or more examples, the machine learning model 164 randomizes the ordering of the training data, visualizes the training data to identify relevant relationships between different variables, identifies any data imbalances, splits the training data into two parts where one part is for training a model and the other part is for validating the trained model, de-duplicates, normalizes, corrects errors in the training data, and so on. The machine learning model 164 implements various machine learning techniques including, for example: K-nearest neighbors; cox proportional hazards model; decision tree learning; association rule learning; neural network (e.g., recurrent neural networks, convolutional neural networks, deep neural networks); inductive programming logic; support vector machines; Bayesian models; REverse Time AttentloN (RETAIN) model; and the like. In another example, the machine learning model 164 leverages one or more classification models trained to classify the training data and/or one or more prediction models trained to predict an outcome based on the training data. Further details of machine learning module are provided below.

In one example, the event prediction module 166 analyzes, in real-time or near real-time, monitored health parameters to determine a type and likelihood of an adverse event occurring with respect to individual 105. In one example, the event prediction module 166 is configured to perform various calculations on processed data to identify a type and determine a likelihood of an adverse event occurring with respect to individual 105. In one embodiment, as discussed elsewhere in the current disclosure, the event prediction module 166 uses a trained machine learning model (e.g., machine learning model 164) to predict probabilities of an adverse event based on health parameters associated with individual 105 and/or responses to health-related questionnaire provided by individual 105.

Health parameters of individual 105 considered include, in some examples, blood pressure, body temperature, skin temperature, heart rate variability, heart rate, resting heart rate, breathing rate, blood glucose levels, oxygen saturation levels, stress levels, or a combination thereof. In another example, the event prediction module 166 accounts for physical activities performed by individual 105 for predetermined time periods, body mass index (BMI) range, prescribed diet, laboratory test values, or a combination thereof. Standards, thresholds, and/or rules for these type of activities, and to which actual performance is compared in some examples, are set by the CPE 130 (e.g., health care provider). In a further example, the event prediction module 166 is configured to account for medication compliance based, at least in part, on adherence to a medication regime prescribed for individual 105.

In one example, the event prediction module 166 identifies a potential adverse event based on a previously determined risk probability, monitored health parameters, and results of one or more pain inquiries. In some examples, the event prediction module 166 allocates more weight to results of pain inquires, conducted via, e.g., electronic questionnaires, than past risk probabilities because such pain inquiry results, in some examples, indicate an imminent, in-progress, or recent occurrence of an adverse event. In some examples, the event prediction module 166 may determine and characterize a likelihood of an adverse event occurring to be: low, moderate, high, severe, or critical; based on a numeric value along a scale (1 to 10; 0 to 100%, etc.); or in a category of a state and/or (medical) industry rating scale (e.g., code 1, 2, or 3, code red, blue, or green, etc.).

In one example, the provisioning list module 168 assigns or reassigns, in real-time or near real-time, positions on one or more provisioning lists for individual 105, relative to positions of other individuals on those provisioning lists, based on a changing risk probability for individual 105 to experience or be subject to an adverse event (e.g., based on the probability of an adverse event for the individual 105 predicted by the machine learning model 164). In practice, this translates into individuals, including individual 105, being assigned positions on respective provisioning lists according to seriousness and urgency of the respective needs of the individuals for limited medical, health, and/or wellness resources that address their respective conditions and/or injuries. In one example, the provisioning list module 168 communicates, via network 140, electronic questionnaires, appointments, provisioning list position changes, and/or removals from provisioning lists directly to individual 105 or through the CPE 130 for review (e.g., by physician or health professionals for confirmation of, for example, list removal) in route to individual 105. In addition, where the individual is first on a provisioning list, or has been removed because a to-be-provisioned appointment, treatment, procedure, or other form of care, has been scheduled, the provisioning list module 168 communicates reminders to individual 105 to schedule a respective form of provisioned care, or a date and time of a scheduled care provisioning. The reminder includes travel directions, customized messages for individual 105, travel time information, etc.

In one example, the user interface module 169 enables a presentation of a graphical user interface (GUI) in the user device 110 and/or one or more computing devices incorporated in the CPE 130. In other examples, the user interface module 169 employs various application programming interfaces (APIs) or other function calls corresponding to an application on the user device 110, thus enabling the display of graphics primitives such as icons, menus, buttons, data entry fields, etc. In another example, the user interface module 169 causes interfacing of guidance information with individual 105 to include, at least in part, one or more annotations, audio messages, video messages, or a combination thereof. In one example, the user interface module 169 comprises a variety of interfaces, for example, interfaces for data input and output devices, referred to as I/O devices, storage devices, and the like. Still further, the user interface module 169 is configured to operate in connection with augmented reality (AR) processing techniques, wherein various applications, graphic elements, and features interact.

In addition, the user interface module 169 is provided to support and provide content incorporated in example care provisioning services described herein. In one example, the user interface module 169 is implemented or embedded in the care provisioning platform 160 or in its functions. In another example, a (actor-specific) respective care provisioning service implemented at the user device 110 or the CPE 130 is supported and/or maintained, directly by the user interface module 169 or by the user interface module 169 through the care provisioning platform 160, to act as a client or agent for the care provisioning platform 160. Accordingly, this example care provisioning service is configured to perform one or more functions associated with the functions of the care provisioning platform 160 by interacting with the care provisioning platform 160 over the network 140.

In one example, the user interface module 169 includes individual and CPE content modules that interact with the care provisioning platform 160 and the data storage systems 180 to respectively supplement or aid in the processing of content information for actor-specific (e.g., individual 105, the CPE 130, actors (doctors, nurses, etc.) within the CPE 130) care provisioning services implemented on various computing devices described herein. Thus, in one or more examples, care provisioning services supported by the user interface module 169 assist in providing the care provisioning platform 160 with health-related information (e.g., activity information that indicates or can be interpreted to determine health conditions of the individuals, contextual information that affect the provisioning list positions for individuals, and a variety of additional information).

Content provided by services maintained or otherwise supported by the user interface module 169, directly or indirectly as described above, include any type of content, such as image content (e.g., pictures), textual content, audio content, video content, etc. In one example, the user interface module 169 provides content that supplements content from sensors or other services incorporated by the user or monitoring devices 110, 120. In one example, the user interface module 169 stores content associated with user interfaces employed by individual and CPE-specific example care provisioning services, and/or other services associated with the care provisioning platform 160. In another example, the user interface module 169 manages access to a central repository of data, stored in one or more stores of the data storage systems 180, for example, and offers a consistent, standard interface to data.

In other examples, the user interface module 169 is configured to support or provide content for: scheduling services, social networking services, content (e.g., audio, video, images, etc.) provisioning services, application services, storage services, contextual information determination services, location-based services, notification services, information (e.g., weather, news, etc.) based services, etc. In other examples, services supported by or including content from the user interface module 169, include various agents or applications such as, but not restricted to, networking applications, multimedia applications, media player applications, camera/imaging applications, software applications, and the like. The above presented modules and components of the care provisioning platform 160 are implemented in hardware, firmware, software, or a combination thereof. As used herein, terms such as “component” or “module” generally encompass hardware and/or software (e.g., that a processor or the like is configured to use to implement associated functionality). Though depicted as a separate entity in FIG. 1, it is contemplated that the care provisioning platform 160 may be implemented in the user device 110, the monitoring device 120, and/or a computing device employed by the CPE 130. The various executions presented herein contemplate any and all arrangements and models.

In the following disclosure, various acts are described as performed or executed by one or more components from FIG. 1, such as the user device 110, the monitoring device 120, the server-side systems 150, a computing device employed by the CPE 130, or one or more combinations thereof. An act performed by a device is considered to be performed by a processor, actuator, or the like associated with that device. Further, it should be understood that in various embodiments, various steps can be added, omitted, and/or rearranged in any suitable manner.

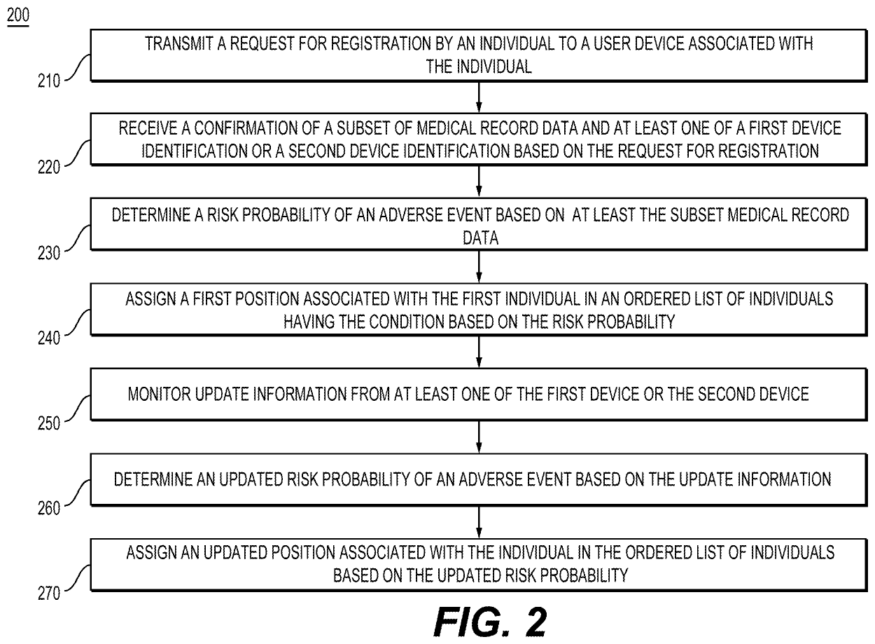

FIG. 2 is a flow chart showing an example process 200 for provisioning care utilizing automated triaging of an electronic care provisioning ordered list (“provisioning list”), according to an aspect of the present disclosure. At step 210, the care provisioning platform 160, in one example, causes a request for registration by individual 105 to be transmitted to the user device 110, or another device associated with individual 105.

Prior to step 210, individual 105 is likely to have developed a condition or suffered an injury and visited a healthcare provider for an evaluation of the condition or injury. Such a healthcare provider determined the condition or injury required some type of specialized care, treatment, and/or surgery for which resources (e.g., hospital beds, doctors, nurses, testing facilities, laboratory personnel and/or equipment) are limited, or otherwise not immediately available for scheduling purposes. As a result, individual 105 was placed on a provisioning list to receive required testing, care, treatment, health-care professional administered medication, or the like.

For example, at or before the point when individual 105 was evaluated, he or she may have suffered an injury to their lower limb and been seen by a healthcare provider that diagnosed individual 105 as having a torn or potentially torn anterior cruciate ligament (ACL). Individual 105 was prescribed a test to confirm or determine a scope of damage to the ligament. Such a test included a magnetic resonance imaging (MRI) scan of the knee. A review of the results of the scan confirmed that the ACL was in fact torn and individual 105 would require surgery to repair their knee. In some healthcare systems, because the injury to individual 105 is not life-threatening, or not so debilitating as to substantially inhibit an ability of the individual to perform normal day to day activities (e.g., work), individual 105 was placed on a provisioning list for knee surgeries. In some example healthcare systems, individual 105 may have been placed on the provisioning list to have the MRI performed, and subsequently added to another provisioning list for the surgery once a review of the MRI was completed.

In some examples, the provisioning list defines an electronic waiting list, or docket, or treatment list, or appointment schedule, or elective surgery schedule, or other types of electronic ordered lists of individuals. Furthermore, individual 105 may have been placed on the provisioning list by a healthcare provider (e.g., doctor, nurse practitioner, nurse, physician's assistant, or the like) or an administrator causing an electronic communication to be transmitted to the care provisioning platform 160. In some examples, the healthcare provider or administrator may have utilized a care provisioning service implemented on a computing device incorporated in the CPE 130, or other CPE according to the present disclosure, to place, or send a request to place, individual 105 on the provisioning list.

Returning to step 210, based on individual 105 being placed on the provisioning list, the care provisioning platform 160 causes the registration request to be transmitted to individual 105. The request asks individual 105 to register for ongoing monitoring using one or more monitoring devices (e.g., the user device 110 and/or the monitoring device 120). The request includes a subset of medical record data (e.g., a portion of data included in the individual's medical records) and asks individual 105 to confirm such data is accurate and/or pertains to the individual. The medical record data referred to herein includes medical records associated with individual 105 and/or any data related to the health/condition/activity of individual 105 collected by the care provisioning platform 160 (or by the data processing module 161 therein). In one example, at step 210, a request portion of a registration process includes transmitting an electronic message to a device specified by individual 105, such as the user device 110, at or some point after the appointment which resulted in individual 105 being placed on the provisioning list. In one or more examples, such an electronic message includes a unique identifier generated by the care provisioning platform 160, and an access invitation. In some examples, the access invitation includes a link to a module of a care provisioning service or a secure website or portal. In another example, the access invitation includes a link to remote electronic treatment options, e.g., online physio for the ACL. The data from that treatment may be fed back into the care provisioning platform 160 to update risk scores, remote treatment interventions, and automated prompts, e.g., non-adherence could lead to higher risk scores, a call from a nurse and remote test for depression, or the like. In other examples, transmitting and delivering the registration request includes using text messaging systems (e.g., SMS) or a patient-level care provisioning service linked to an integrated cloud environment incorporated in, or incorporating, the care provisioning platform 160.

During the registration process, the care provisioning platform 160 presents to, or causes to present to, individual 150 a list of monitoring devices specific to the condition, injury, and/or care, treatment, consultation, surgery, therapy, or service needed. For example, with a torn ACL, some portion of remote monitoring performed is accomplished, in some examples, using an accelerometer of the user device 110, where the user device 110 includes a mobile phone for example. Accordingly, the user device 110 is presented in the list of monitoring devices as part of an example registration process. Therefore, in some examples, individual 105 receives, via a text message, push notification, email, or like notification accessible from a mobile phone, an invitation to enroll, link, or otherwise use their mobile phone (e.g., the user device 110 or some other personal electronic device they carry on their person) to track and send movement data from a respective accelerometer or other motion detecting component for the mobile phone. In one example, accelerometer data is collected such that a position of the individual in space is determinable by, for example, the care provisioning platform 160. In other examples, this type of data access (e.g., mobile phone accelerometer data) enables the care provisioning platform 160, and potentially other systems of the server-side systems 150, to determine the manner in which individual 105 is moving, such as, e.g., how much individual 105 is moving, movement patterns exhibited by individual 105, the speed by which individual 105 is moving, and the like.

In another example, individual 105 may be waiting for vascular surgery. As a result, a list of devices presented to individual likely includes the monitoring device 120 or another monitoring device, such as a wearable device or other type of device, which monitors blood glucose levels. A blood glucose monitor, in some areas of the world, is an example of a monitoring device that is not incorporated in mobile phones or otherwise not possessed by some individuals that register with an example care provisioning platform such as the care provisioning platform 160. In these instances, in addition to the inclusion of an enrollment process as described above, example registration processes according to present disclosure include dispatch or distribution processes based on or in response to selections or assignment of such a monitoring device to an individual such as individual 105. In some embodiments, the care provisioning platform 160 initiates a process of requesting or directing vendors, insurance providers, healthcare providers, a CPE (such as the CPE 130), or the like to send a monitoring device associated with a condition, injury, or service needed for individual 105. Thus, the care provisioning platform 160 automatically orders (via entries to the online form) the blood glucose monitor and dispatch the blood glucose monitor to individual 105 waiting for vascular surgery.

At step 220, the care provisioning platform 160 receives a confirmation of a subset of medical record data and at least one of a first device identification or a second device identification based on the request for registration. More specifically, in some examples, based on individual 105, accepting (e.g., clicking a link) the access invitation, the care provisioning platform 160 presents to, or causes to present to, individual 105 an electronic form with basic electronic medical records (EMR) data and information related to the needed care, treatment, surgery, or the like. In some examples, a secure cloud-hosted server contains all EMR data, but only select details are shown in the electronic form. EMR data included in an electronic form are presented for purposes of verification by individual 105. More specifically, individual 105 is given an option to confirm the EMR details before being presented with an option to select one or more monitoring devices for ongoing monitoring.

In other examples, at step 220, example registration processes include presenting individual 105 with a series of questions related to how individual 105 perceives their current state of health. In some examples, this includes requesting ratings for pain, when pain is felt, and/or how often a particular pain is felt. Other questions presented in the registration process implemented with steps 210 and 220, in some examples, include how often does individual 105 go to the bathroom, have trouble going to the bathroom, hours of sleep individual 105 normally gets, or the like.

In some examples, a verification process described above is coupled with a monitoring device registration process in step 220. In addition, the registration process includes a permission request to exchange communications with healthcare providers and administrators required by certain regulatory organizations/government entities for transmitting medical information or medical related information electronically.

According to some aspects of the present disclosure, monitoring devices include mobile phones, smartwatches, single use testing devices, dedicated health monitors (e.g., wearable devices) that track heart rate, steps, motion, blood/oxygen saturation, blood glucose levels, ketone levels, variable heart rate, and the like. For example, with a respective accelerometer, a mobile phone can provide a monitoring device configured to measure movement by individual 105 and potentially detect abnormal movement patterns (e.g., falls, limping, seizure, etc.). In some examples, step 220 includes receiving at least one of an identification of a first device corresponding to a user device, such as the user device 110, or an identification of a second device corresponding to a monitoring device, such as the monitoring device 120. Furthermore, step 220 includes receiving from individual 105 permission to access, initiate, cause to operate, and/or receive data from services, components (e.g., cameras, sensors, accelerometers, etc.) incorporated in the user device 110 and/or the monitoring device 120.

At step 230, the care provisioning platform 160 determines a risk probability of an adverse event (“risk probability”) associated with individual 105 based on at least the subset of medical record data. In one embodiment, the care provisioning platform 160 determines the risk probability based on the subset of medical record data. In other embodiments, the care provisioning platform 160 determines the risk probability based on some portion (e.g., that is different from the subset that is presented to individual 105 for verification) or all of the medical record data. In some examples, the care provisioning platform 160 is configured to utilize machine learning to determine risk probabilities of adverse events occurring to individual 105, as a result of not receiving/having to wait for care, treatments, and/or procedures associated with a provisioning list to which individual 105 is assigned a position. In turn, the care provisioning platform 160 is configured to order/assign a position to individual 105 on a provisioning list, according to a respective risk probability in step 240.

In some examples, the care provisioning platform 160 determines a probability of an adverse event (risk probability), and an updated probability of an adverse event (updated risk probability) described in more detail with respect to step 260, using a trained machine learning model generated by training a machine learning model on a plurality of patient conditions and corresponding probabilities of an adverse event. Subsequently, example machine learning models according the to the present disclosure are trained on portions, combinations of portions, and/or an entirety of the plurality of patient (individual) conditions and corresponding probabilities of an adverse events to output a probability distribution over the plurality of patient (individual) conditions, each probability in the probability distribution indicating a likelihood an individual, such as individual 105, will experience an adverse event due to a corresponding patient condition (and having to remain on a provisioning list).

In some examples, the care provisioning platform 160 is configured to utilize and/or implement machine learning models, such as a REverse Time AttentloN (RETAIN) model, to generate risk probabilities for condition or injury-specific adverse events (e.g., falls in individuals awaiting hip surgery). In one embodiment, at baseline (e.g., at a point of placement on, or automated addition to, example provisioning lists described herein), example care provisioning services and platforms are configured determine risk probabilities based on EMR data (e.g., age, sex, weight, history of injuries, history of falls, number of visits/amount of time since accident, emergency, identification of provisioning list-associated condition or injury, etc.).

In some embodiments, different machine learning models are generated to tailor to varying circumstances under which the individuals in need of the disclosed care provisioning service may be situated. For example, training data for one example model relative to another example model varies from standpoints of health conditions, required care, resource availability, types of adverse events, and the like. In other examples, variation of training data is due to: a (general) location of an individual; a healthcare system or systems an individual has access to; modes of transportation available to and distances of medical facilities from an individual; a type of health insurance possessed or available to an individual; recognized standards of care specific to where an individual lives or is otherwise located; and/or recognized levels of skill associated with healthcare professionals and providers specific to where an individual lives or is located and/or is available to that individual.

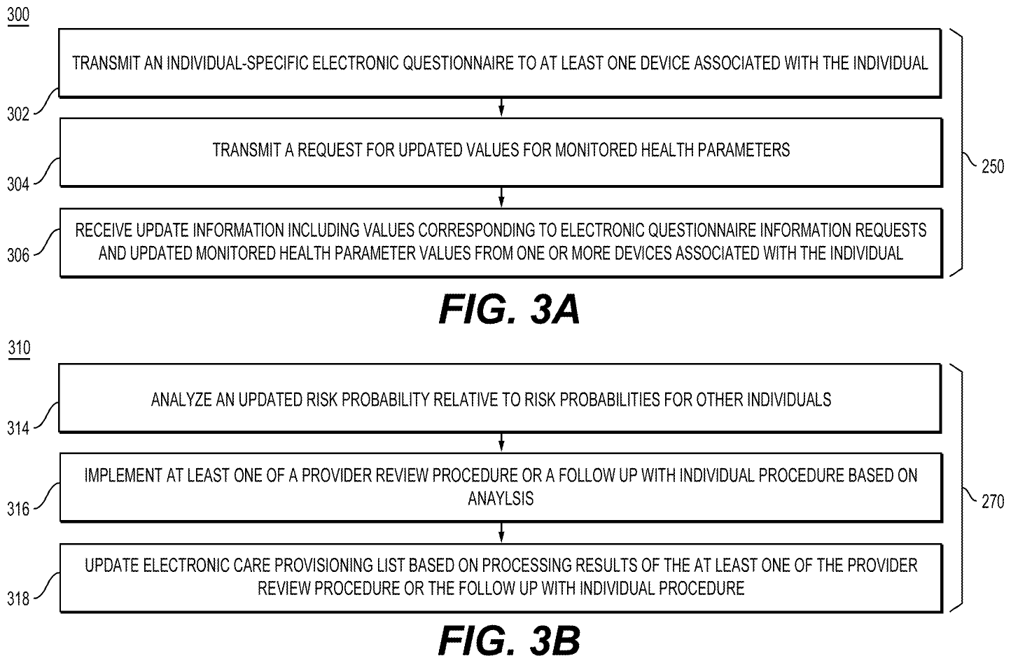

At step 240, the care provisioning platform 160 assigns individual 105 a position in an electronic ordered list of individuals having the same or a similar condition or injury as individual 105, based on the risk probability determined for individual 105 in step 230. Aspects of step 240 are discussed in more detail with respect to the example process 310 of FIG. 3B.

At step 250, update information from a first device (e.g., the user device 110) and/or a second device (e.g., the monitoring device 120) is monitored. In some examples, provisioning list-specific remote monitoring data are continuously captured via the registered monitoring devices described herein. In addition to continuous monitoring, systems and methods described herein are configured to provide the care provisioning platform 160 with the types of information that reflect the characteristics of face to face appointments between patients and healthcare providers. In some examples, step 250 includes regular electronic questionnaires automatically being sent to individuals, such as individual 105, via the registered devices specified during the registration process. The care provisioning platform 160 provides the electronic questionnaires via: text message; a care provisioning service implemented on the registered device as a dedicated application or web application; a portal accessible from registered devices and/or non-registered devices (e.g., a laptop or tablet not involved in monitoring activities); or communication platforms implementing health care services/applications. Aspects of step 250 directed toward electronic questionnaires are discussed in more detail with respect to the example process 300 of FIG. 3A.

At step 260, the care provisioning platform 160 determines an updated risk probability of an adverse event based on the update information for individual 105. Step 260 includes similar processes as step 230 with the addition of processes for integrating the update information (e.g., activity/condition/health data captured by the registered devices and/or responses to the questionnaires submitted from the registered devices) into the determination of a (updated) risk probability. More specifically, remote monitoring and questionnaire data are integrated into example machine learning models and respective risk probabilities for individuals, such as individual 105, are updated on a recurring (e.g., daily, weekly, biweekly) or as-needed (e.g., upon receipt of update information from registered devices) basis. In some examples, accessing, integrating, and/or updating data collection schedules are modified based on a previously determined risk probability. For example, care provisioning platforms are configured to increase a frequency for one or all of the processes based on an increase in a risk probability, a change in a monitored health parameter (e.g., blood glucose level), or a response (answer value) of one or more questions included in a questionnaire.

In addition, the care provisioning platform 160 is configured to generate or cause to generate dashboards or various types of user interfaces, and cause the same to be presented to individual 105 via a display of, for example, the user device 110 (e.g., a mobile phone), the monitoring device 120, other registered devices, or an unregistered device implementing a web application. In some examples, content presented in dashboards and other user interfaces generated by the care provisioning platform 160 include reports (static and interactive), graphs, progressions, and/or playbacks of remote monitoring data. In turn, individuals, such as individual 105, are able to keep track of their progress and potentially alter their behavior in ways that may enable them to qualify for specific needed care, treatments, and/or procedures representing the reason for being placed on an example provisioning list.

For example, an individual 105 requires a procedure requiring individual 105 have a lower body mass index (BMI) than at the time individual 105 was placed on a provisioning list. The required BMI is presented in a display, in some examples, as a goal or metric in a graphical user interface (“GUI”) generated by an example care provisioning service being implemented on a registered device (e.g., the user device 110, the monitoring device 120) or through a web application. The BMI goal in some examples provides a control to which every monitored activity performed by individual 105 is compared or evaluated as to a respective value relative to accomplishing the BMI goal. In turn, individual 105 is encouraged to increase his or her movement as individual 105 sees a gap between the goal and his or her current BMI decline.

At step 270, the care provisioning platform 160 assigns individual 105 an updated position in a respective provisioning list of individuals having the same or a similar condition or injury as individual 105, based on the updated risk probability determined for the individual in step 260. Aspects of step 270 are discussed in more detail with respect to the example process 310 of FIG. 3B.

FIG. 3A is a flow chart showing an example process for monitoring individuals assigned positions in provisioning lists as part of process 200 of FIG. 2, according to an aspect of the present disclosure. At step 302, the care provisioning platform 160 transmits an individual-specific electronic questionnaire to at least one device associated with individual 105. In some examples, electronic questionnaires according to the present disclosure include multiple types of health-related questions and answer input formats. In substantially all instances, example questionnaires include questions specific to one or more of: a condition or injury that was a primary reason for individual 105 being placed on a respective provisioning list; a health factor, parameter, or vital related to or impacted by a condition or injury suffered by individual 105; a health condition (e.g., last time individual 105 ate, used the bathroom, required assistance to eat or use the bathroom) relevant to a fitness of individual 105 to receive a treatment or procedure; and resources and/or medical professional and/or healthcare providers required for the care, treatment, procedure, consultation, and/or testing individual 105 is waiting for by being included on the respective provisioning list.

According to some aspects of the present disclosure, example questions include in questionnaires focus on specific details regarding any pain an individual 105 is experiencing while being included in a respective provisioning list and awaiting care, a procedure, a treatment, consultation, or a diagnostic associated with the respective provisioning list. Example details included in questions posited to individuals, such as individual 105, include: a level of pain, on a specified scale (e.g., 1 to 10), individual 105 is experiencing; when was the last time individual 105 experienced pain; how long does the pain last or has lasted; how often does individual 105 experience the pain; a degree, on a specified scale (e.g., 1 to 10), to which the pain inhibits an ability to complete normal day to day activities such as those required for employment; and/or what time of day does individual 105 experience the pain.

The “truth” or “accuracy” of self-determined pain scores could be evaluated using machine learning and, where suspected exaggeration occurs, a different line of questioning or virtual consultation with a nurse could be put to the patient. In this way, remote monitoring, questioning, interventions, and ultimately end-treatment offered are individualized to patients and continuously updated based on modelling of the data in their EMR, monitoring devices, and questionnaire responses. Related to “end-treatment”, e.g., ACL surgery, all the data collected might also impact the delivery of that. For example, someone with a high risk for sepsis might be automatically be booked in for a longer post treatment hospital stay for infection monitoring and increased anti-biotics. In conventional approaches, beds and treatments are generally not tailored to individual needs.

In some examples, formats for answers to questionnaire questions are configured by the care provisioning platform 160 according to a method, process, and/or machine learning model implemented to generate risk probabilities used to assign individuals respective ordered positions on example provisioning lists. In some examples, answer formats for a single questionnaire include binary data input types (e.g., Y/N, Yes/No, I/O), numeric data input types (e.g., 0 to 10, 1 to 5, 0 to 100%), tertiary non-numeric data input types (e.g., high/medium/low, all/some/none, yes/maybe/no), and or typed-in text input.

In some examples, answers that must be selected from binary, numeric, or tertiary answer options (e.g., via drop-down boxes) are used/processed directly and used by the care provisioning platform 160 in determining risk probabilities. An answer value for each of these answer types is capable of being given a corresponding numeric value (0 or 1; 1 to 10; 1 or 2 or 3) or weight. The care provisioning platform 160 is configured to use said values as input for example algorithms, models, machine learning models discussed therein.

In other examples, the care provisioning platform 160 is configured to generate questions requiring typed-in responses. In examples, such questions are generated based on responses to questions specifying a binary, numeric, or tertiary data type as answer value options. An example care provisioning service provided by the user interface module 169 and implemented on a computing device, through which a questionnaire is presented and answers for individual 105 are input, incorporates a word parsing and/or type of character recognition module or service configured to process text/typed-in answers. Such parsing and recognition services, under the direction of example care provisioning services and/or platforms, identify flag words. Responses including certain keywords are used as input for determining a risk probability in some examples, and/or flagged for review by a healthcare professional or provider in other examples. Evaluation by such a healthcare professional or provider includes, in some examples, selecting answer options provided in certain formats (e.g., binary, numeric) and processed as input for determining a risk probability associated with an individual 105. Text answers by individual 105 or text, binary, or numeric answers from the CPE 130 providers, in other examples, are normalized and input into a machine learning model, along with monitoring data and other questionnaire data, for determining a risk probability for individual 105. Individuals might have risks for a range of events not all directly related to their treatment, e.g., patients on a lower limb waiting list but with a high probability of a mental health breakdown could be offered video-call based counselling. And as above, the range of risk probabilities for situations like surgical complications will be used to design and deliver a specific end-treatment like a surgery (e.g., glue versus sutures, local versus general anesthetic, length of post-treatment stay, or the like).

In other examples, a risk probability assigned to an individual 105 comprises probability sub-components. For example, a first probability sub-component corresponds to a risk probability based on monitoring and questionnaire response data. Typed-in answers, by individual 105 or the CPE 130, are separated from health monitoring data and questionnaire response data, and used to generate a separate second risk probability component. In some examples, information from external sources, such as data associated with intermediate remedial measures (e.g., pre-surgery physical therapy, medication, counseling, etc.), is processed by the care provisioning platform 160 as a second or third probability sub-component of a risk probability determined for individual 105.

At step 304, the care provisioning platform 160 transmits one or more requests for updated values for monitored health parameters. In some examples, the care provisioning platform 160 is configured to access and/or request monitored health parameter data as well as treatment data and/or medical records from providers of remedial measures. In addition, the care provisioning platform 160 is configured to process information from these sources to update risk probabilities and generate questions for electronic questionnaires that account for any improvements or setbacks reflected in the information.

At step 306, the care provisioning platform 160 receives update information including values corresponding to electronic questionnaire information requests and updated monitored health parameter values from one or more devices associated with individual 105. As used herein, the term “real-time,” “real time,” and “substantially real-time” are used to refer to processing, content generation, and information presentation performed within time constraints. For example, as described herein, real-time tracking, collection, processing, question generation, and question inclusion in example electronic questionnaires can be performed as an event triggering that performance occurs, without a perceivable delay after the occurrence of the event.

In some examples, the care provisioning platform 160 implements a series of rules and recognizes answers to one or a combination of pain related questions included in an example questionnaire as indicative of an emergency or potential emergency situation. According to aspects of the present disclosure, the care provisioning platform 160 is configured to make these recognitions in real-time and present additional, and/or generate subsequent pain-related questions for which certain series of answers will be recognized as corresponding to an emergency situation.

For example, individual 105, as included in a provisioning list for knee surgery, provides information during a registration period that leads to the care provisioning platform 160 determining, based on medical record data associated with individual 105, that the individual has high blood pressure and is heavier than an ideal weight for healthy individuals of the same height. Although injured knees requiring surgical repair and cardiac arrest are not recognized as comorbid conditions, based on answers by individual 105 to pain-related questions, the care provisioning platform 160 generates a series of questions that reveals individual 105 is having a heart attack. An example questionnaire posits the question of how much activity or time, on a presented scale (e.g., 1 to 10, 0 to 100%, etc.), has individual 105 spent on their feet. In some examples, the event prediction module 166 of the care provisioning platform 160 is configured to recognize a potential emergency situation that could result in cardiac arrest from: (1) an answer to this question, coupled with (2) a response to a question of a level of pain in the knee currently experienced being over a predetermined value, further coupled with (3) a heart rate or blood/oxygen saturation level provided by a continuous monitoring device being at or above a certain value as a potential emergency situation that could result in cardiac arrested. As a result, in some examples, subsequent questions presented in an electronic questionnaire will ask about conditions that are not related to a knee of individual 105 or knee pain therein. In some examples, such other questions will inquire as to whether individual 105 is feeling pain or tightness in their chest, how long has the pain or tightness been persisting, how painful, on a presented scale (e.g., 1 to 10), is the pain or tightness.

In still other examples, example electronic questionnaires include questions related to whether or not an individual 105 is currently receiving some type of remedial type of care, treatment, or procedures for a condition or injury that caused individual 105 to be placed on a provisioning list. In some examples, such remedial measures include physical therapy, prescription medication, some form of counseling, wellness programs, or the like that are not included in a group of limited resources associated with care provisioning electronic lists according to the present disclosure.

In still other examples, the care provisioning platform 160 may be configured to perform individualised and automatically initiated remote care (e.g., online physio, depression testing, or the like) and questioning based on EMR data, monitoring and machine learning risk prediction data. In still other examples, the care provisioning platform 160 may be configured to perform individualised and automatic treatment planning and scheduling based on said data, e.g., glue vs stitches, in versus out patient, length of stay, type of antibiotics, et al. For instance, the care provisioning platform 160 may be configured to automatically initiate remote care and questioning based on the medical record data and machine learning risk prediction data, or automatically perform treatment planning and scheduling based on the medical record data.

FIG. 3B is a flow chart showing an example process for assigning individuals updated positions in provisioning lists as part of process 200 of FIG. 2, according to an aspect of the present disclosure. At step 314, the care provisioning platform 160 analyzes an updated risk probability for individual 105 relative to risk probabilities for other individuals in the provisioning list.

Example care provisioning platforms according to the present disclosure are configured to reorder provisioning lists automatically based on outputs from machine learning models, such as RETAIN outputs, corresponding to risk probabilities, as well as probability sub-components mentioned above. More specifically, individuals associated with higher risk probabilities for adverse events (e.g., falls, cardiac arrest, mental breakdown, etc.) are positioned on provisioning lists closer to a first position than other individuals. In other examples, an individual, such as individual 105, is grouped with a first group of individuals assigned a first value (such as a number one (1) or higher) for a designation, which is “lower” or “higher” relative to a second value assigned to a second group of individuals having lower risk probabilities than the first group.

According to aspects of the present disclosure, certain individuals, for example those positioned on a provisioning list closer to one (1), will receive care, a procedure, or a treatment they are “in line for” before other individuals, because they are, in essence, closer to “the front of the line” than those other individuals.

As noted above, adverse events that individual 105 is considered as being at risk of experiencing are in many examples related, to varying degrees, to an injury, condition, or health-related issue serving as a reason, at least in part, for individual 105 being placed on a provisioning list. According to aspects of the present disclosure, responses to questionnaires and recorded values for monitored parameters for individual 105, identified as a first individual, are provided as input for one or more machine learning models, and/or other risk assessments. Output from these models and assessments provide a risk probability, a composite risk probability, and/or probability sub-component for individual 105. The care provisioning platform 160 compares the determined risk probability for individual 105 to corresponding probabilities associated with other individuals listed in the provisioning list including individual 105, and determines the position on the provisioning list for individual 105 based on these comparisons. For example, individual 105 is assigned a first position in an example provisioning list for a hip surgery. At the first position, a first number of individuals are listed before individual 105 and are therefore slated to receive hip surgery before individual 105. However, after being placed on the list, implementations of the systems and methods described herein, for example step 260, result in a determination that a probability individual 105 will experience an adverse event is greater, e.g., individual 105 is more at risk to experience an adverse event, than at a time when a risk probability associated with the first position was determined. In this example, some if not all risk probabilities for the first number of individuals stay the same, and at least some of those probabilities are lower than the updated risk probability for individual 105. Accordingly, the updated positioned for individual 105 is lower (e.g., closer to the top of the list) than the previous position. As a further result, a second number individuals listed before individual 105 at the updated position is less than the first number of individuals.

In some examples, care provisioning platforms according to the present disclosure are configured to transmit a notification to a provider of care for a condition associated with a provisioning list that includes individual 105. Referring to the hip surgery example above, the updated position for individual 105 is a first position or in a first group of individuals on the hip surgery provisioning list. A notification is transmitted in some examples, to a provider that identifies individual 105 as having the lowest position in the ordered list and a next recipient of the care associated with the condition. The notification to the provider includes contact information for individual 105 in some examples, so the provider can reach out to individual 105. In addition, individual 105 receives a notification that they are next in some examples.

In other examples, a difference between risk and update risk probabilities, or position and updated positions, is greater than a predetermined threshold. In such examples, care provisioning platforms described herein are configured to transmit a notification to a provider of care for a condition associated with a provisioning list regarding these situations. In some examples, the notification specifies the difference between the risk and updated risk probabilities and/or the position and updated position, and includes contact information for an individual so the provider can reach out to the individual. Notifications to providers mentioned above are presented, in some examples, in CPE versions of a care provisioning service according to some aspects of the present disclosure.

Determining risk probabilities and positioning/re-positioning individuals, such as individual 105, on provisioning lists are processes implemented by the care provisioning platform 160, in some examples, within integrated cloud environments that contain individual 105-specific EMR, monitoring, questionnaire, RETAIN, staff schedule, and bed availability data. An individual, such as individual 105, with the highest risk probability is scheduled into a next available slot for care, treatment, procedure, and/or counseling that defines the limited resource an individual 105 was placed on a particular provisioning list for.

In some examples, individual 105 is automatically informed that “it is their turn” via communication transmitted to a registered device via a text message (e.g., SMS) and/or a notification provided to the user device 110, the monitoring device 120, or through a web application implemented on a registered or unregistered device. The care provisioning platform 160 is configured to send such messages and notifications. In some examples, such a notification can include alert, push notification, vibration or alarm selected by individual 105, an automated phone call or text message, or the like.

One of ordinary skill will readily appreciate the benefits of assigning positions on electronic provisioning lists according to risk probabilities. As discussed above, individuals, such as individual 105, with the highest risks of experiencing adverse events will have access to one or more health/medical resources that address their respective conditions or injuries, before individuals with lower risk probabilities for experiencing adverse events. In practice, this results in individuals with the most serious and most urgent limited resource needs being set up to receive those resources before other individuals with less serious and less urgent needs for the same resources. As a result, automated care provisioning processes implemented by the care provisioning platform 160 accurately, reliably, and rapidly (real-time) allocate, or allow access to, limited resources based on need. This significant benefit is delivered to those with the most serious and urgent needs, without requiring manual processes that involve reviews: (1) of voluminous medical records that are not updated in real-time, and do not include questionnaire response data and therefore lack direct information from individuals equivalent to information obtained in face to face patient/provider interactions/appointments; (2) by medical and healthcare professionals and providers that are part of the limited resources that individuals must be placed in queues to have access to. Thus, methods and systems described herein are configured to provide individuals with the most serious and urgent needs with prioritized access to limited resources, while at the same time making those limited resources more available to more individuals, such as individual 105, by lessening the amount or degree to which these limited resources must be expended on administrative tasks and/or non-emergency patient triage.

At step 316, the care provisioning platform 160 implements at least one of a provider review procedure, or a follow up with an individual procedure, based on the analysis of the updated position for individual 105 in step 314. In some examples, situations warranting a provider review procedure or a follow up with an individual procedure correspond to those situations where an individual no longer needs to be included on a provisioning list or is experiencing an emergency. Thus, the care provisioning platform is configured to issue notifications in step 316 to identify these situations and direct a provider and/or an individual's attention to the individual's health and questionnaire information for evaluation and further action (e.g., list removal, emergency care, etc.).

According to other aspects of the present disclosure, methods and systems directed toward example care provisioning platforms and services described herein also increase availability of limited resources to individuals, such as individual 105, having the highest risk probabilities for experiencing adverse event/most serious and urgent needs. More specifically, example systems and methods described herein lessen a load on healthcare providers by removing individuals from provisioning lists that see improvements with their respective conditions or injuries to the extent that limited resources individual 105 was placed on an example provisioning lists for, are no longer needed.

For example, in some healthcare systems, it is not uncommon for individuals to unnecessarily remain for substantial periods of time on waiting lists to receive limited resources (e.g., care, treatment, procedures, counseling, or the like). On the other hand, example methods and systems described herein mitigate these types of scenarios by providing regular automatic prompts to individuals, such as individual 105, via texts messages (e.g., SMS), mobile phone delivered notifications, and/or features of example care provisioning services. Such prompts direct individuals to electronic questionnaires in an uncomplicated manner.

A similarly uncomplicated process for submitting electronic questionnaire responses follows. In turn individuals are enabled to provide up-to-date information regarding respective conditions or injuries that, once processed, could reflect care is no longer needed. Accordingly, once these responses are submitted to, for example, an integrated cloud environment incorporated in or configured to provide example care provisioning platforms described herein, individuals are removed from provisioning lists in a qualified manner. As a result, systems and methods described herein are configured to reduce the cases of individuals unnecessarily occupying positions on lists for care for extended periods of time.

In addition, example care provisioning services and platforms described herein deliver, tangentially, an added benefit of symptoms experienced by individuals being reduced through remote clinical monitoring and presentation of associated data. As individuals, such as individual 105, feel better over a time period when they are waiting for a limited resource as described above, a number of individuals who do not elect to seek out a treatment receive care, or have a procedure corresponding to a limited resource providing the reason they were originally placed on a provisioning list.

Regarding elections to be removed from provisioning lists, example care provisioning systems and platforms according to the present disclosure facilitate qualified elections to not seek out treatment, receive care, or have a procedure that is limited in nature. In some examples, such elections are made and processed through controlled and automatically recorded communications between individuals (e.g., via mobile device) and healthcare providers. Example provisioning platforms are configured to guard against individuals that may superficially feel better (e.g., from a placebo effect), making decisions that ultimately have negative consequences for the individuals' health (and/or healthcare provider liability). Care provisioning platforms according to the present disclosure are configured to implement processes through a series of electronic prompts and individual 105/healthcare provider communications. Furthermore, provisioning service/platform-guided workflows that incorporate these prompts and communications are configured to initiate and record status verifications regarding individuals. Such status verifications are conducted through short in-person evaluations, analyses of health parameter monitoring data, and/or review of biometric test results by, or otherwise facilitated as part of, the above-mentioned workflows. Such processes tangibly, via e.g., electronic memorializing, ensure efficacy, safety, fairness, and a way to address concerns about individual 105 self-determination versus provider determinations. Machine learning can be implemented to identify those experiencing placebo and those potentially holding on to their place unnecessarily.

At step 318, the care provisioning platform 160 updates a provisioning list based on processing results of the at least one of the provider review procedure or the follow up with individual procedure.

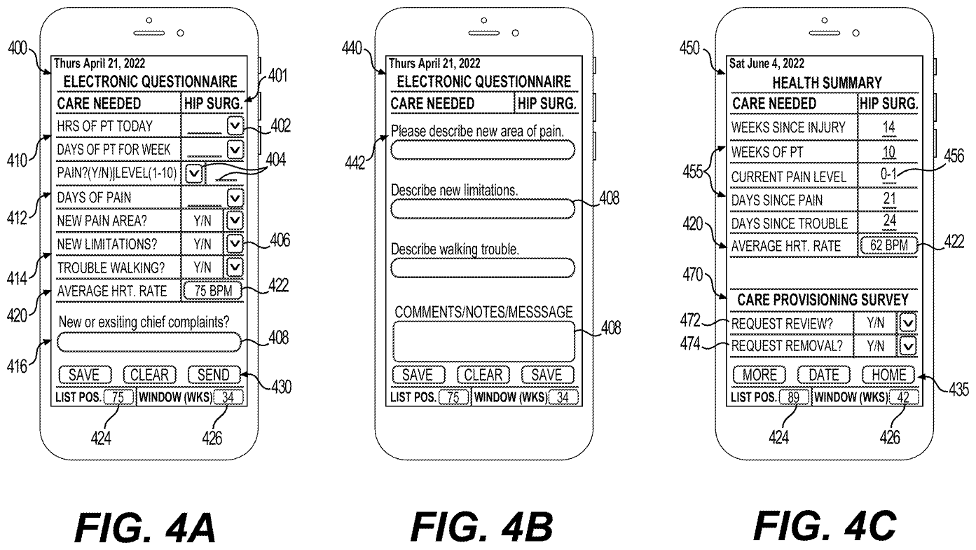

FIGS. 4A through 4C are example user interface diagrams that illustrate an example information flow related to example electronic questionnaires, according to an aspect of the present disclosure. FIGS. 4A and 4B depict illustrations of example GUIs of an electronic questionnaire. In particular, FIG. 4A illustrates an example initial electronic questionnaire GUI 400 that includes a root condition or injury identification field 401 and a table of questions 410-416 and answer input fields generated, in some examples, by a care provisioning service according to the present disclosure. The questions presented, in some examples, are part of one or more question categories including remedial measures 410, pain 412, new limitations 414, and condition or injury-related complaints 416. Answer input fields, as shown include numeric 402, combination 404, binary 404, and typed-response 408. The electronic questionnaire GUI 400 further includes monitored health parameters 420 and monitored health parameter values 422 providing one, averaged, and/or mean values for specified parameters. In addition, provisioning list position information 424 and projected wait times 426 that correspond to a position specified in the position information 424 are incorporated in the electronic questionnaire GUI 400. Furthermore, an individual presented with the electronic questionnaire GUI 400 is able to save, clear, and/or send entered information first menu options 430.

FIG. 4B illustrates a derivative electronic questionnaire GUI 440 according to an aspects of the present disclosure. More specifically, the derivative GUI 440 is generated, for example by a care provisioning service, in response to answers provided in the initial electronic questionnaire GUI 400. More specifically, the derivative questionnaire GUI 400 includes follow-up questions 442 that an individual, such as individual 105, provides typed-response 408 answers. As discussed above, questions provided in such GUIs depend from, and request specific information with respect to, answers provided in the initial questionnaire GUI 400 and the results from risk prediction machine learning models.

FIG. 4C depicts an illustration of an exemplary health summary GUI 450, according to one or more examples. As illustrated, the health summary GUI 450 includes historical health categories 455 that relate to the condition or injury specified in the condition or injury field 401. Recorded values 456 for the historical health categories 455 represent current, average over time, total deviation from diagnosis to current day, and most recent health statistics for an individual. Like the initial questionnaire GUI 400, the health summary GUI 450 includes monitored health parameters 420 and monitored health parameter values 422 that in some examples, characterize values for a given monitored health parameter over a total period of time the parameter has been monitored. Provisioning list position information 424 and projected wait times 426 that correspond to a position specified in the position information 424 are also incorporated in the health summary GUI 450. Second menu options 435 may be used to view more historical health categories 455 as well as values for historical health categories 455 on different dates.

The health summary GUI 450, depending on a status (health) of an individual with respect to a condition or injury that caused the individual to be placed on a provision list, also includes a care provisioning survey section 470. An individual may utilize options in this section to a make first request 472 for a healthcare professional to review the health summary, or to make a second request 474 for the individual to be removed from a respective provisioning list. The first request 472 is configured to allow an individual to request review from a provider for the purposes of moving the individual closer to a first position in a provisioning list, or to be removed from the provisioning list in some examples.

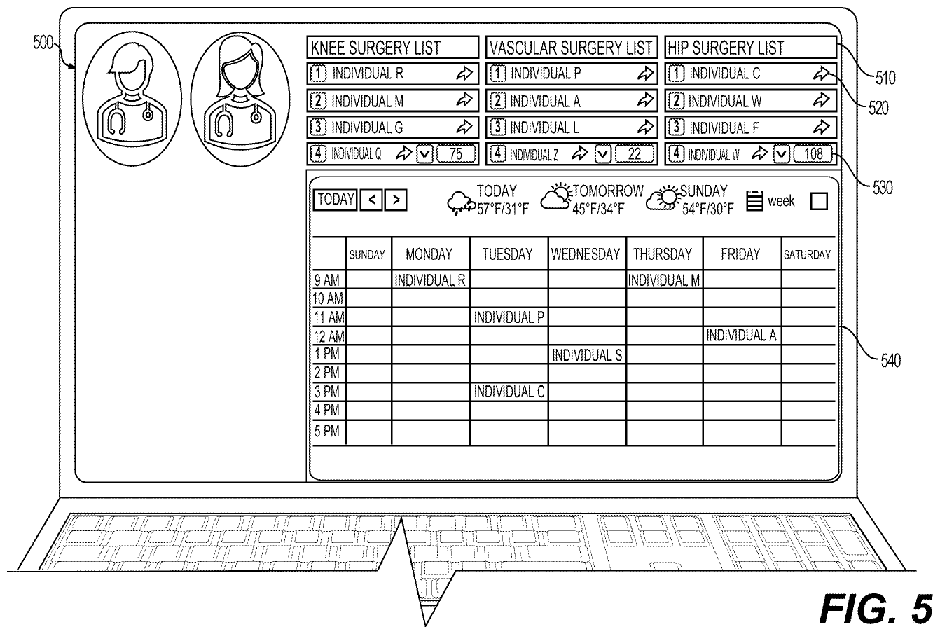

FIG. 5 is an example user interface 500 that illustrates example provisioning lists 510, according to aspects of the disclosure. The provisioning lists 510 as displayed include a treatment, procedure, or care for which individuals have been placed on respective lists. In addition, each provisioning list 510 may include an option 520 that may be selected for each listed individual that when selected, provides particular information regarding that individual. Such information may include injury information as well as monitored health parameter and electronic questionnaire data for a selected individual. As shown, each of the provisioning lists is present with an indicator 530 that informs a user as to a total number of individuals included in one of the provisioning lists. One of ordinary skill in the art will recognize that an individual may be placed on one or more provisioning lists. In addition to the features mentioned above, the user interface 500 of FIG. 5 includes a calendar 540 that informs as to what individuals are scheduled for (provisioned) appointments to receive care, treatment, or a procedure associated with one of the provisioning lists 510 displayed by the user interface 500.

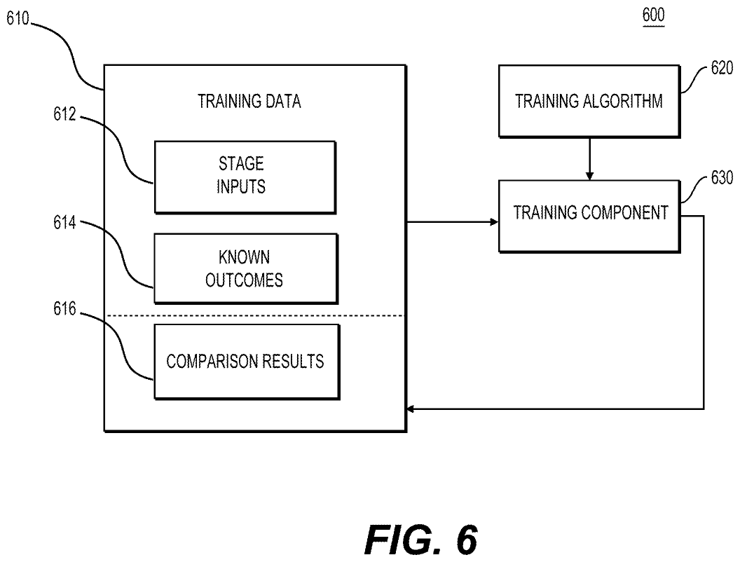

One or more implementations disclosed herein include and/or may be implemented using machine learning models (e.g., the machine learning model 164). For example, one or more of the modules of the care provisioning platform 160 may be implemented using a machine learning model and/or may be used to train the machine learning model (e.g., training module 162). A given machine learning model may be trained using the data flow 600 of FIG. 6. Training data 610 includes one or more of stage inputs 612 and known outcomes 614 related to the machine learning model to be trained. The stage inputs 612 may be from any applicable source including text, visual representations, data, values, comparisons, stage outputs, e.g., one or more outputs from one or more steps from FIGS. 2-3B. The known outcomes 614 may be included for the machine learning models generated based on supervised or semi-supervised training. An unsupervised machine learning model may not be trained using known outcomes 614. Known outcomes 614 includes known or desired outputs for future inputs similar to or in the same category as stage inputs 612 that do not have corresponding known outputs.

The training data 610 and a training algorithm 620, e.g., one or more of the modules implemented using the machine learning model and/or may be used to train the machine learning model, may be provided to a training component 630 that may apply the training data 610 to the training algorithm 620 to generate the machine learning model. According to an implementation, the training component 630 may be provided comparison results 616 that compare a previous output of the corresponding machine learning model to apply the previous result to re-train the machine learning model. The comparison results 616 may be used by training component 630 to update the corresponding machine learning model. The training algorithm 620 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, or the like.

The machine learning model used herein may be trained and/or used by adjusting one or more weights and/or one or more layers of the machine learning model. For example, during training, a given weight may be adjusted (e.g., increased, decreased, removed) based on training data or input data. Similarly, a layer may be updated, added, or removed based on training data/and or input data. The resulting outputs may be adjusted based on the adjusted weights and/or layers.

In general, any process or operation discussed in this disclosure that is understood to be computer-implementable, such as the process illustrated in FIGS. 2, 3, and 3B may be performed by one or more processors of a computer system as described herein. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, includes one or more computing devices. One or more processors of a computer system may be included in a single computing device or distributed among a plurality of computing devices. One or more processors of a computer system may be connected to a data storage device. A memory of the computer system includes the respective memory of each computing device of the plurality of computing devices.

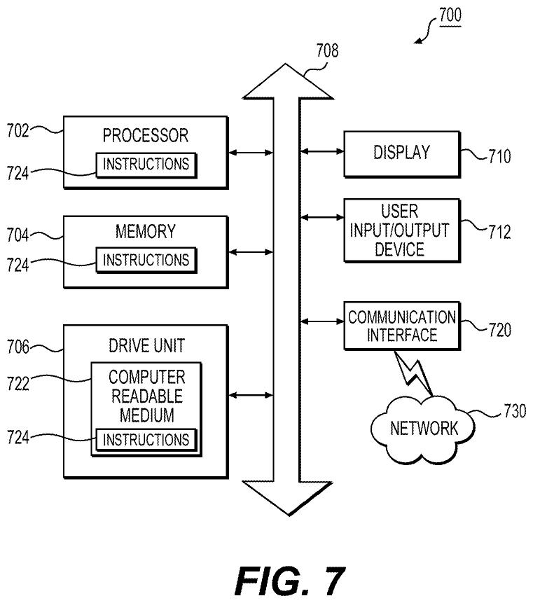

FIG. 7 illustrates an implementation of a computer system that may execute techniques presented herein. The computer system 700 can include a set of instructions that can be executed to cause the computer system 700 to perform any one or more of the methods or computer-based functions disclosed herein. The computer system 700 may operate as a standalone device or may be connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining,” “analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data (e.g., from registers and/or memory to transform that electronic data into other electronic data that (e.g., may be stored in registers and/or memory. A “computer,” a “computing machine,” a “computing platform,” a “computing device,” or a “server” includes one or more processors.

In a networked deployment, the computer system 700 may operate in the capacity of a server or as a client user computer in a server-client user network environment, or as a peer computer system in a peer-to-peer (or distributed) network environment. The computer system 700 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 700 can be implemented using electronic devices that provide voice, video, or data communication. Further, while a computer system 700 is illustrated as a single system, the term “system” shall also be taken to include any collection of systems or sub-systems that individually or jointly execute a set, or multiple sets, of instructions to perform one or more computer functions.

As illustrated in FIG. 7, the computer system 700 includes a processor 702 such as a central processing unit (CPU), a graphics processing unit (GPU), or both. The processor 702 may be a component in a variety of systems. For example, the processor 702 may be part of a standard personal computer or a workstation. The processor 702 may be one or more general processors, digital signal processors, application specific integrated circuits, field programmable gate arrays, servers, networks, digital circuits, analog circuits, combinations thereof, or other now known or later developed devices for analyzing and processing data. The processor 702 may implement a software program, such as code generated manually (i.e., programmed).

The computer system 700 includes a memory 704 that can communicate via a bus 708. The memory 704 may be a main memory, a static memory, or a dynamic memory. The memory 704 includes, but is not limited to, computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 704 includes a cache or random-access memory for the processor 702. In alternative implementations, the memory 704 is separate from the processor 702, such as a cache memory of a processor, the system memory, or other memory. The memory 704 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 704 is operable to store instructions executable by the processor 702. The functions, acts or tasks illustrated in the figures or described herein may be performed by the processor 702 executing the instructions stored in the memory 704. The functions, acts or tasks are independent of the particular type of instructions set, storage media, processor or processing strategy and may be performed by software, hardware, integrated circuits, firm-ware, micro-code and the like, operating alone or in combination. Likewise, processing strategies includes multiprocessing, multitasking, parallel processing and the like.

As shown, the computer system 700 may further include a display 710, such as a liquid crystal display (LCD), an organic light emitting diode (OLED), a flat panel display, a solid-state display, a cathode ray tube (CRT), a projector, a printer or other now known or later developed display device for outputting determined information. The display 710 may act as an interface for the user to see the functioning of the processor 702, or specifically as an interface with the software stored in the memory 704 or in the drive unit 706.

Additionally, or alternatively, the computer system 700 includes an input/output device 712 configured to allow a user to interact with any of the components of computer system 700. The input/output device 712 may be a number pad, a keyboard, or a cursor control device, such as a mouse, or a joystick, touch screen display, remote control, or any other device operative to interact with the computer system 700.

The computer system 700 may also or alternatively include drive unit 706 implemented as a disk or optical drive. The drive unit 706 includes a computer-readable medium 722 in which one or more sets of instructions 724, e.g., software, can be embedded. Further, instructions 724 may embody one or more of the methods or logic as described herein. The instructions 724 may reside completely or partially within the memory 704 and/or within the processor 702 during execution by the computer system 700. The memory 704 and the processor 702 also includes computer-readable media as discussed above.

In some systems, a computer-readable medium 722 includes instructions 724 or receives and executes instructions 724 responsive to a propagated signal so that a device connected to a network 730 can communicate voice, video, audio, images, or any other data over the network 730. Further, the instructions 724 may be transmitted or received over the network 730 via a communication port or interface 720, and/or using a bus 708. The communication port or interface 720 may be a part of the processor 702 or may be a separate component. The communication port or interface 720 may be created in software or may be a physical connection in hardware. The communication port or interface 720 may be configured to connect with a network 730, external media, the display 710, or any other components in computer system 700, or combinations thereof. The connection with the network 730 may be a physical connection, such as a wired Ethernet connection or may be established wirelessly as discussed below. Likewise, the additional connections with other components of the computer system 700 may be physical connections or may be established wirelessly. The network 730 may alternatively be directly connected to a bus 708.

While the computer-readable medium 722 is shown to be a single medium, the term “computer-readable medium” includes a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 722 may be non-transitory and may be tangible.

The computer-readable medium 722 can include a solid-state memory such as a memory card or other package that houses one or more non-volatile read-only memories. The computer-readable medium 722 can be a random-access memory or other volatile re-writable memory. Additionally, or alternatively, the computer-readable medium 722 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 700 may be connected to a network 730. The network 730 may define one or more networks including wired or wireless networks. The wireless network may be a cellular telephone network, an 602.11, 602.16, 602.20, or WiMAX network. Further, such networks include a public network, such as the Internet, a private network, such as an intranet, or combinations thereof, and may utilize a variety of networking protocols now available or later developed including, but not limited to TCP/IP based networking protocols. The network 730 includes wide area networks (WAN), such as the Internet, local area networks (LAN), campus area networks, metropolitan area networks, a direct connection such as through a Universal Serial Bus (USB) port, or any other networks that may allow for data communication. The network 730 may be configured to couple one computing device to another computing device to enable communication of data between the devices. The network 730 may generally be enabled to employ any form of machine-readable media for communicating information from one device to another. The network 730 includes communication methods by which information may travel between computing devices. The network 730 may be divided into sub-networks. The sub-networks may allow access to all of the other components connected thereto or the sub-networks may restrict access between the components. The network 730 may be regarded as a public or private network connection and includes, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component/object distributed processing, and parallel processing. Alternatively, virtual computer system processing can be constructed to implement one or more of the methods or functionalities as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure may be implemented using any appropriate techniques for implementing the functionality described herein. The disclosure is not limited to any particular programming language or operating system.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures, and techniques have not been shown in detail in order not to obscure an understanding of this description.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects.
Example 1. A computer-implemented method includes transmitting, by one or more processors, a request for registration by an individual having a condition to a user device associated with the individual, the request including a subset of data associated with the individual; receiving, by the one or more processors, a confirmation of the subset of data and at least one of a first device identification associated with a first device or a second device identification associated with a second device based on the request; determining, by the one or more processors, a risk probability of an adverse event based on at least the subset of data; assigning, by the one or more processors, a position associated with the individual in an ordered list of individuals having the condition based on the risk probability; monitoring, by the one or more processors, update information from at least one of the first device or the second device; determining, by the one or more processors, an updated risk probability of an adverse event based on the update information; and assigning, by the one or more processors, an updated position associated with the individual in the ordered list of individuals based on the updated risk probability.
Example 2. The computer-implemented method of Example 1, wherein the update information includes measurements of movements by the individual from an accelerometer.
Example 3. The computer-implemented method of any of the preceding examples, wherein the update information includes blood glucose measurements for the individual from a blood glucose monitoring device.
Example 4. The computer-implemented method of any of the preceding examples, wherein the first device includes a communications device specified by the individual for receiving individualized update requests.
Example 5. The computer-implemented method of Example 4, wherein monitoring the update information comprises: transmitting, by the one or more processors, a data object to the first device; and receiving, by the one or more processors, the update information from the first device; wherein the update information includes one or more values corresponding to one or more information requests specified in the data object.
Example 6. The computer-implemented method of any of the preceding examples, wherein the first device includes a communications device and the second device includes a health monitoring device configured to monitor health parameters, the health parameters including at least one of movements, vitals, or blood glucose levels of the individual.
Example 7. The computer-implemented method of Example 6, wherein monitoring the update information comprises: transmitting, by the one or more processors, a data object to the first device; transmitting, by the one or more processors, a request for updated values for the monitored health parameters from the second device; receiving, by the one or more processors, the update information from the first device and the second device, wherein the update information includes values corresponding to information requests specified in the data object and the updated values for the monitored health parameters.
Example 8. The computer-implemented method of any of the preceding examples, further comprising determining, by the one or more processors, a monitoring schedule based on the probability of an adverse event.
Example 9. The computer-implemented method of Example 8, further comprising modifying the monitoring schedule based on the updated probability of an adverse event.
Example 10. The computer-implemented method of any of the preceding examples, further comprising removing the individual from the ordered list or adding the individual to another list based on the update information.
Example 11. The computer-implemented method of any of the preceding examples, wherein a first number of individuals are listed before the individual at the position, wherein an increase of the updated risk probability relative to the risk probability indicates the individual is more at risk to experience an adverse event than at a time corresponding to a determination of the risk probability, wherein the updated position is lower than the position, wherein the updated position indicates care associated with the condition will be provisioned to a second number individuals before the individual, and wherein the second number of individuals is less than the first number of individuals.
Example 12. The computer-implemented method of any of the preceding examples, further comprising transmitting a notification to a provider of care associated with the condition based on the ordered list, wherein the notification identifies one of the individuals included in the ordered list as having a lowest position in the ordered list and a next recipient of the care associated with the condition.
Example 13. The computer-implemented method of any of the preceding examples, further comprising transmitting a notification to one individual from the individuals based on a respective position in the ordered list, wherein the notification identifies the one individual as a next recipient of care associated with the condition.
Example 14. The computer-implemented method of any of the preceding examples, wherein the probability of an adverse event and the updated probability of an adverse event are determined using a trained machine learning model, wherein the trained machine learning model is generated by: receiving, as training data, a plurality of individual conditions and corresponding probabilities of an adverse event; and training a machine learning model based on at least a portion of the plurality of individual conditions and corresponding probabilities of an adverse event to output a probability distribution over the plurality of individual conditions, each probability in the probability distribution indicating a likelihood an individual will experience an adverse event due to a corresponding individual condition.
Example 15. The computer-implemented method of Example 1, wherein the computer-implemented method further includes automatically initiating remote care and questioning based on the data and machine learning risk prediction data; or automatically performing treatment planning and scheduling based on the data.
Example 16. A system includes one or more processors; and at least one memory storing instructions that, when executed by the one or more processors, cause the one or more processors to perform operations including: transmitting a request for registration by an individual having a condition to a user device associated with the individual, the request including a subset of data associated with the individual; receiving a confirmation of the subset of data and at least one of a first device identification associated with a first device or a second device identification associated with a second device based on the request; determining a risk probability of an adverse event based on at least the subset of the data; assigning a position associated with the first individual in an ordered list of individuals having the condition based on the risk probability; monitoring update information from at least one of the first device or the second device; determining an updated probability of an adverse event based on the update information; and updating the position associated with the individual in the ordered list based on the updated probability of an adverse event.
Example 17. The system of Example 16, wherein the first device includes a communications device specified by the individual for receiving individualized update requests.
Example 18. The system of any of Examples 16-17, wherein monitoring the update information comprises: transmitting a data object to the first device; and receiving the update information from the first device, wherein the update information includes one or more values corresponding to one or more information requests specified in the data object.
Example 19. The system of any of Examples 16-18, the operations further comprising removing the individual from the ordered list based on the update information
Example 20. A non-transitory computer readable medium stores instructions which, when executed by one or more processors, cause the one or more processors to perform operations comprising: transmitting a request for registration by an individual having a condition to a user device associated with the individual, the request including a subset of data associated with the individual; receiving a confirmation of the subset of data and at least one of a first device identification associated with a first device or a second device identification associated with a second device based on the request; determining a risk probability of an adverse event based on the data; assigning a position associated with the individual in an ordered list of individuals having the condition based on the risk probability; monitoring update information from at least one of the first device or the second device; determining an updated probability of an adverse event based on the update information; and updating the position associated with the individual in the ordered list based on the updated probability of an adverse event.

What is claimed is:

1. A computer-implemented method comprising:
transmitting, by one or more processors, a request for registration by an individual having a condition to a user device associated with the individual, the request including a subset of data associated with the individual;
receiving, by the one or more processors, a confirmation of the subset of data and at least one of a first device identification associated with a first device or a second device identification associated with a second device based on the request;
determining, by the one or more processors, a risk probability of an adverse event based on at least the subset of data;
assigning, by the one or more processors, a position associated with the individual in an ordered list of individuals having the condition based on the risk probability;
monitoring, by the one or more processors, update information from at least one of the first device or the second device;
determining, by the one or more processors, an updated risk probability of an adverse event based on the update information; and
assigning, by the one or more processors, an updated position associated with the individual in the ordered list of individuals based on the updated risk probability.

2. The computer-implemented method of claim 1, wherein the update information includes measurements of movements by the individual from an accelerometer.

3. The computer-implemented method of claim 1, wherein the update information includes blood glucose measurements for the individual from a blood glucose monitoring device.

4. The computer-implemented method of claim 1, wherein the first device includes a communications device specified by the individual for receiving individualized update requests.

5. The computer-implemented method of claim 4, wherein monitoring the update information comprises:
transmitting, by the one or more processors, a data object to the first device; and
receiving, by the one or more processors, the update information from the first device;
wherein the update information includes one or more values corresponding to one or more information requests specified in the data object.

6. The computer-implemented method of claim 1, wherein the first device includes a communications device and the second device includes a health monitoring device configured to monitor health parameters, the health parameters including at least one of movements, vitals, or blood glucose levels of the individual.

7. The computer-implemented method of claim 6, wherein monitoring the update information comprises:
transmitting, by the one or more processors, a data object to the first device;
transmitting, by the one or more processors, a request for updated values for the monitored health parameters from the second device;
receiving, by the one or more processors, the update information from the first device and the second device,
wherein the update information includes values corresponding to information requests specified in the data object and the updated values for the monitored health parameters.

8. The computer-implemented method of claim 1, further comprising determining, by the one or more processors, a monitoring schedule based on the probability of an adverse event.

9. The computer-implemented method of claim 8, further comprising modifying the monitoring schedule based on the updated probability of an adverse event.

10. The computer-implemented method of claim 1, further comprising removing the individual from the ordered list or adding the individual to another list based on the update information.

11. The computer-implemented method of claim 1,
wherein a first number of individuals are listed before the individual at the position,
wherein an increase of the updated risk probability relative to the risk probability indicates the individual is more at risk to experience an adverse event than at a time corresponding to a determination of the risk probability,
wherein the updated position is lower than the position,
wherein the updated position indicates care associated with the condition will be provisioned to a second number individuals before the individual, and
wherein the second number of individuals is less than the first number of individuals.

12. The computer-implemented method of claim 1, further comprising transmitting a notification to a provider system associated with the condition based on the ordered list, wherein the notification identifies one of the individuals included in the ordered list as having a lowest position in the ordered list and a next recipient of the care associated with the condition.

13. The computer-implemented method of claim 1, further comprising transmitting a notification to one individual from the individuals based on a respective position in the ordered list, wherein the notification identifies the one individual as a next recipient of care associated with the condition.

14. The computer-implemented method of claim 1, wherein the probability of an adverse event and the updated probability of an adverse event are determined using a trained machine learning model, wherein the trained machine learning model is generated by:
receiving, as training data, a plurality of individual conditions and corresponding probabilities of an adverse event; and
training a machine learning model based on at least a portion of the plurality of individual conditions and corresponding probabilities of an adverse event to output a probability distribution over the plurality of individual conditions, each probability in the probability distribution indicating a likelihood an individual will experience an adverse event due to a corresponding individual condition.

15. The computer-implemented method of claim 1, further comprising:
automatically initiating remote care and questioning based on the data and machine learning risk prediction data; or
automatically performing treatment planning and scheduling based on the data.

16. A system comprising:
one or more processors; and
at least one memory storing instructions that, when executed by the one or more processors, cause the one or more processors to perform operations including:
transmitting a request for registration by an individual having a condition to a user device associated with the individual, the request including a subset of data associated with the individual;
receiving a confirmation of the subset of data and at least one of a first device identification associated with a first device or a second device identification associated with a second device based on the request;
determining a risk probability of an adverse event based on at least the subset of the data;
assigning a position associated with the first individual in an ordered list of individuals having the condition based on the risk probability;
monitoring update information from at least one of the first device or the second device;
determining an updated probability of an adverse event based on the update information; and
updating the position associated with the individual in the ordered list based on the updated probability of an adverse event.

17. The system of claim 16, wherein the first device includes a communications device specified by the individual for receiving individualized update requests.

18. The system of claim 16, wherein monitoring the update information comprises:
transmitting a data object to the first device; and
receiving the update information from the first device,
wherein the update information includes one or more values corresponding to one or more information requests specified in the data object.

19. The system of claim 16, the operations further comprising removing the individual from the ordered list based on the update information.

20. A non-transitory computer readable medium storing instructions which, when executed by one or more processors, cause the one or more processors to perform operations comprising:
transmitting a request for registration by an individual having a condition to a user device associated with the individual, the request including a subset of data associated with the individual;
receiving a confirmation of the subset of data and at least one of a first device identification associated with a first device or a second device identification associated with a second device based on the request;
determining a risk probability of an adverse event based on at least the subset of data;
assigning a position associated with the individual in an ordered list of individuals having the condition based on the risk probability;
monitoring update information from at least one of the first device or the second device;
determining an updated probability of an adverse event based on the update information; and
updating the position associated with the individual in the ordered list based on the updated probability of an adverse event.
